(19) United States
(12) Patent Application Publication
(10) Pub. No.: US 2009/0259246 A1
(43) Pub. Date: Oct. 15, 2009

(54) Intragastric Volume-Occupying Device

(76) Inventors: Sherif Eskaros, Elkton, MD (US); David C. Everson, Chesapeake City, MD (US); Richard A. Bucher, Lincoln University, PA (US); Cedomila Ristic-Lehmann, Bear, DE (US); Melissa Sherman, Kennet Square, PA (US)

Correspondence Address: GORE ENTERPRISE HOLDINGS, INC., 551 PAPER MILL ROAD, P. O. BOX 9206, NEWARK, DE, 19714-9206, US

(21) Appl. No.: 12/102,389

(22) Filed: Apr. 14, 2008

(51) Int. Cl. A61B 17/00; A61F 2/00; A61B 19/00

(52) U.S. Cl. 606/192

(57) ABSTRACT

An intragastric device having a non-degradable porous membrane enclosing a material which expands upon contact with fluid, and causes the volume of the device to increase is described. The intragastric device is useful for managing satiety and weight loss.

### FIELD OF THE INVENTION

The present invention relates to a device that aids in managing weight reduction and satiety.

### BACKGROUND OF THE INVENTION

Obesity is a major health problem in developed countries. In the United States, the complications of obesity affect nearly one in five individuals at an annual cost of approximately $40 billion.

Currently there are a number of Bariatric Procedures for treating obesity, including surgical procedures such as Roux-en-Y Gastric Bypass, Biliopancreatic Diversion (BPD), Gastric Banding, and Gastroplasty. These procedures fall under two categories—malabsorptive and restrictive.

Several medical devices that are designed to mimic malabsorptive procedures are disclosed in: U.S. Patent Appl. Nos. 2007/0032879 A1; US 2005/0096750 A1; US2006/0293742A1; US 2006/0020247 A1; US 2004/0117031 A1; US 2007/0010794 A1; US 2007/0010864 A1; and U.S. Pat. No. 7,122,058 B2; U.S. Pat. No. 7,025,791 B2; U.S. Pat. No. 7,037,344 B2; U.S. Pat. No. 4,641,653 A; U.S. Pat. No. 4,501,264; U.S. Pat. No. 4,763,653 A; U.S. Pat. No. 7,314,489 B2; and U.S. Pat. No. 7,316,716 B2.

Another group of devices are designed to occupy stomach volume to induce the feeling of “satiety” or “fullness.” Intragastric volume-occupying devices provide the patient a feeling of satiety after having eaten only small amounts of food. Thus, the caloric intake is diminished while the subject is satisfied with a feeling of fullness. Clinical use of intragastric balloons has been ongoing for several years, and its success in the treatment of certain individuals with morbid obesity is well accepted. Volume-occupying devices for use in obesity reduction were developed in the late 1970's and early 1980's. These early designs had multiple complications that caused them not to gain widespread acceptance at the time. Newer designs were developed in the late 1980's, and have led to their wider acceptance in European clinics.

U.S. Pat. No. 4,133,315 discloses an apparatus for reducing obesity comprising an inflatable, elastomeric bag and tube combination. According to the '315 patent, the bag can be inserted into the patient's stomach by swallowing. The end of the attached tube distal to the bag remains in the patient's mouth. A second tube is snaked through the nasal cavity and into the patient's mouth. The tube ends located in the patient's mouth are connected to form a continuous tube for fluid communication through the patient's nose to the bag. Alternatively, the bag can be implanted by a gastrotomy procedure. The bag is inflated through the tube to a desired degree before the patient eats so that the desire for food is reduced. After the patient has eaten, the bag is deflated. As taught by the '315 patent, the tube extends out of the patient's nose or abdominal cavity throughout the course of treatment.

U.S. Pat. Nos. 5,259,399, 5,234,454, and 6,454,785 disclose intragastric volume-occupying devices for weight control that must be implanted surgically. U.S. Pat. Nos. 4,416,267; 4,485,805; 4,607,618; 4,694,827, 4,723,547; 4,739,758; 4,899,747, German Patent DE 35 40 936, and European Patent No. 246,999 relate to intragastric, volume-occupying devices for weight control that can be inserted endoscopically. Of these, U.S. Pat. Nos. 4,416,267; 4,694,827; 4,739,758, and 4,899,747 relate to balloons whose surface is contoured in a certain way to achieve a desired end. In the '267 and '747 patents, the balloon is torus-shaped with a flared central opening to facilitate passage of solids and liquids through the stomach cavity. The balloon of the '827 patent has a plurality of smooth-surfaced convex protrusions. The protrusions reduce the amount of surface area, which contacts the stomach wall, thereby reducing the deleterious effects resulting from excessive contact with the gastric mucosa. The protrusions also define channels between the balloon and stomach wall through which solids and liquids may pass. The balloon of the '758 patent has blisters on its periphery that prevent it from seating tightly against the cardia or pylorus.

U.S. Pat. No. 5,129,915 relates to an intragastric balloon that is intended to be swallowed and that inflates automatically under the effect of temperature. The '915 patent discusses three ways that an intragastric balloon might be inflated by a change in temperature. A composition comprising a solid acid and non-toxic carbonate or bicarbonate is separated from water by a coating of chocolate, cocoa paste or cocoa butter that melts at body temperature. Alternatively, citric acid and an alkaline bicarbonate coated with non-toxic vegetable or animal fat melting at body temperature and which placed in the presence of water, would produce the same result. Lastly, the solid acid and non-toxic carbonate or bicarbonate are isolated from water by an isolation pouch of low-strength synthetic material which it will suffice to break immediately before swallowing the balloon. Breaking the isolation pouches causes the acid, carbonate, or bicarbonate and water to mix and the balloon to begin to expand immediately. A drawback of thermal triggering of inflation as suggested by the '915 patent is that it does not afford the degree of control and reproducibility of the timing of inflation that is desirable and necessary in a safe self-inflating intragastric balloon.

The present invention provides a minimally invasive intragastric device for inducing satiety and for weight reduction applications. According to the present invention, the intragastric device expands from a first volume (hereinafter, initial shape) to a second volume (hereinafter, expanded volume).

### SUMMARY OF THE INVENTION

The present invention provides an intragastric device comprising a non-degradable porous membrane enclosing an expandable material which swells upon contact with fluid, and causes the volume of the device to increase.

### DESCRIPTION OF THE DRAWINGS

FIG. 1 is a perspective view of an intragastric device prior to introduction into a patient.

FIG. 2 is a perspective view of an intragastric device in an expanded state.

FIG. 3 is an intragastric device with a deactivation means on the exterior of the device.

FIG. 4 shows a device packed into a delivery capsule.

FIGS. 5A and 5B show an intragastric device formed from two pieces of non-degradable porous membrane enclosing an expandable material.

FIG. 6 shows an aspect of the intragastric device wherein the membrane is configured to allow the device to have a ball or tomato like form as the final shape after expansion.

FIG. 7 shows an aspect of the intragastric device wherein the membrane is configured to allow the device to have spiral form as the final shape after expansion.

### DETAILED DESCRIPTION OF THE INVENTION

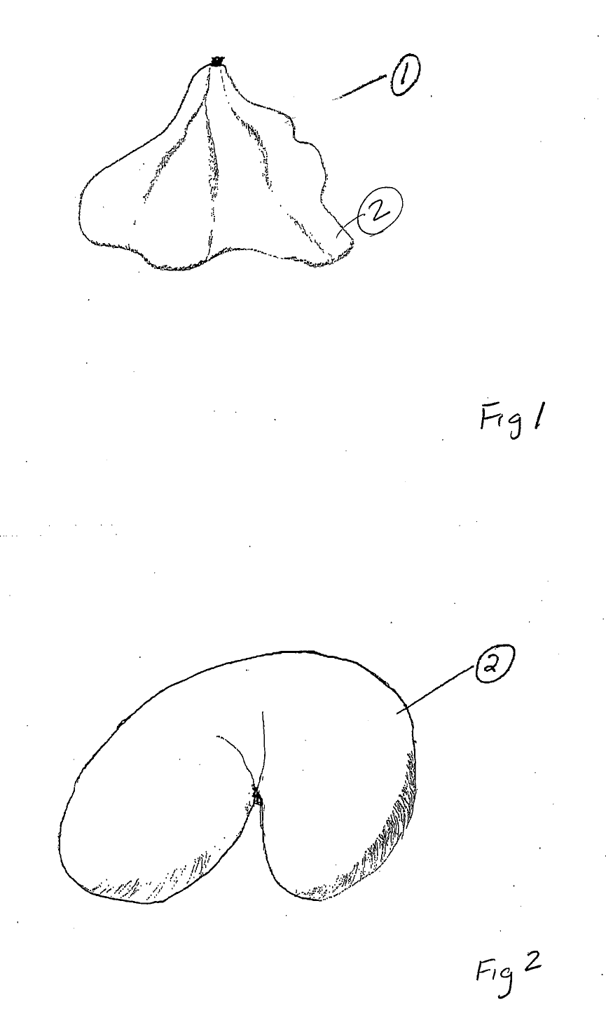

Referring to the drawings in which like reference numbers represent like or corresponding elements in the drawings, FIGS. 1 and 2 illustrate one particular embodiment of an intragastric device incorporating features of the present invention. An intragastric device (1) comprises at least a non-degradable porous membrane (2) enclosing an expandable material (3) which expands upon contact with a hydrating fluid, and causes the volume of the intragastric device (1) to increase to an expanded volume forming a final shape. The hydrating fluid is natural gastric fluid or an ingestible fluid including, but not limited to, aqueous liquids, basic and acidic solutions, and the like. In yet a further embodiment, the expanded volume has at least 5 times, preferably 100-1000 times the volume of initial shape. The intragastric device can be delivered to a patient concurrently with a hydrating fluid. The hydrating fluid may have a pH of between 1.5 and 9.

The intragastric device (1) may be sized to be delivered orally, endoscopically, or via other minimally invasive methods. As shown in FIG. 4, the device may be formed or packed into delivery components (4) including components such as gel capsules, coatings, dip coats, sprays, bandings or other such forms to aid in delivery of the device. One example for minimally invasive delivery includes sizing the device to be delivered orally via a swallowable capsule.

In a further embodiment the initial shape of the intragastric device may have a volume between 0.1 ml to 28 ml, preferably 0.5 ml to 10 ml In yet a further embodiment the intragastric device is provided in the various initial shapes including, but not limited to, kidney-shaped, oblong, cylindrical, oval, rectangular, ellipse, triangle, conical, trapezoidal, star-like, pear-like, umbrella-like, butterfly, bow-tie, snare-like, coil, helical, spiral, doughnut, or spherical. In yet another embodiment, the intragastric device is delivered endoscopically. In one embodiment, the expanded volume of the intragastric device may have a volume between 20 ml to 1500 ml, preferably 100 ml to 500 ml.

The device (1) comprises biologically inert and compatible materials for medical use. By “inert” it is meant that the device is compatible with the body in which it is used, and has no anticipated chemical action within the body.

The expandable material is able to increase in volume to occupy additional space after activation with liquid or other activating means. For instance, it may be desirable to achieve a final volume capacity with the activated expandable material of at least 100 ml/g using water as an activating means. A radio-opaque contrast, imaging agents such as metals, and/or dyes may be incorporated into the device for fluoroscopic image tracking. The contrast agents may be dispersed or mixed with the expandable material, or may be incorporated as a part of the membrane, coatings or seals. It may also be desirable to form all or part of the device with radiopaque materials to aid in imaging.

Examples of suitable contrast agents include, but are not limited to, BaSO₄, BiO₃, Au, Pt, PtIr, W, I.

The device (1), after the implantation, expands in the stomach to induce a feeling of satiety after eating smaller amounts of food. The device may remain in the stomach in its approximate final form for a desired period of time prior to disruption of the membrane or seal of the device, which will facilitate volume decrease of the device so that the device may then pass naturally out of the body for elimination, or if desired, the device may be removed endoscopically.

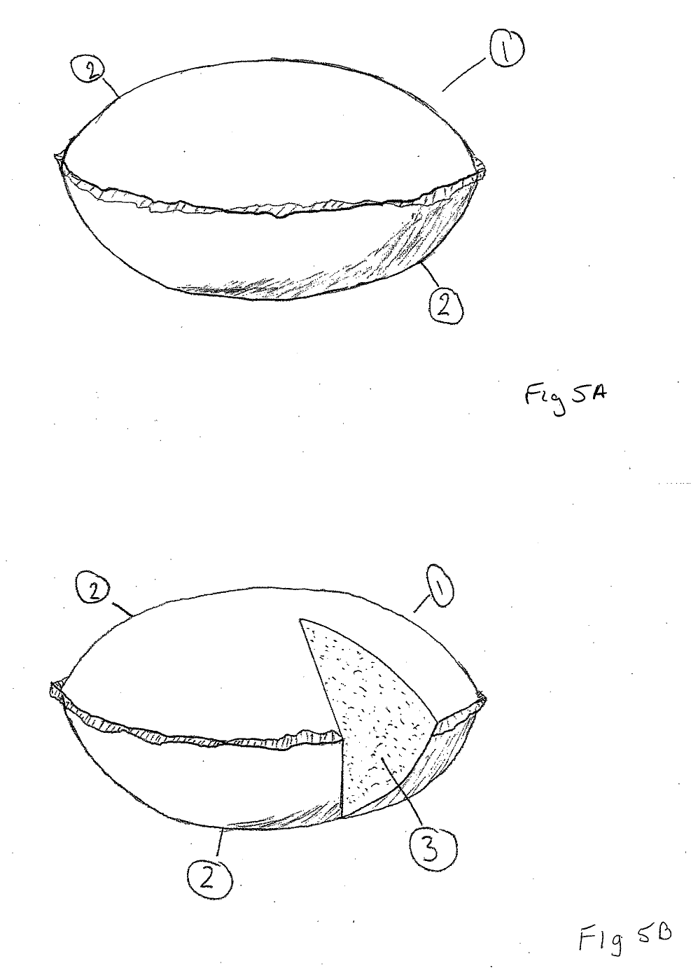

The non-degradable porous membrane (2) is not disruptable under physiological conditions, such as body temperature, stomach peristalsis, stomach acid, ingested foods, and the like. Additionally, the non-degradable porous membrane is selected based upon the duration of use of the device in situ. The membrane which covers the expandable material may be formed from one piece or from multiple pieces. When multiple membrane pieces are used, the pieces may be similar or different in properties such as density, porosity, shape, size, thickness, mechanical strength, and the like. As shown in FIGS. 5A and 5B, two pieces of membrane may be joined to form the outer shell or sack with holds the expandable material (3). The intragastric device is intended to remain in the stomach of a user for an ample period of time such that either satiety or weight loss is achieved by the user. The period of time can vary depending upon the individual circumstances; however, in most cases, it is desirable to achieve placement of the device in the stomach for at least 7 days. It may be desirable to achieve a placement of the device in the stomach for more extended periods of time. Suitable materials for the non-degradable porous membrane include, but are not limited to, water permeable polymers, liquid permeable polymers, expanded polytetrafluoroethylene (ePTFE), fluoropolymers, silicone, elastomers, resins, polyurethanes, poly vinyl alcohols, expanded ultrahigh molecular weight polyethylenes, and the like. The membranes may also be chosen for desired media such as imparting a differing liquid permeability in basic pH fluids as compared to acidic media. In one aspect of the invention, the non-degradable membrane can be a fibrillated membrane such as ePTFE. In another aspect, the intragastric device may have a non-degradable porous membrane that maintains a constant relative pore size during expansion of the material.

In certain applications, a non-degradable fibrillated membrane is chosen as the non-porous membrane to provide strength and expansion properties desirable upon swelling of the expandable material to increase the device volume.

The non-degradable porous membrane may be further coated with various materials to introduce desired characteristics to the membrane. The membrane may be coated on all or a portion of its surface. Further the membrane may be coated on one or both sides, with like or unlike coating. For instance, an ePTFE membrane may be coated with a glutaraldehyde cross-linked PVA coating to render it hydrophilic. Other coatings may be used: to induce lipophilic properties, hydrophobic properties, and drug elution properties; polyelectrolytes; low surface energy coatings to minimize irritation of the stomach wall; fluoropolymer coatings, or other coatings to modulate surface energy and biocompatibility.

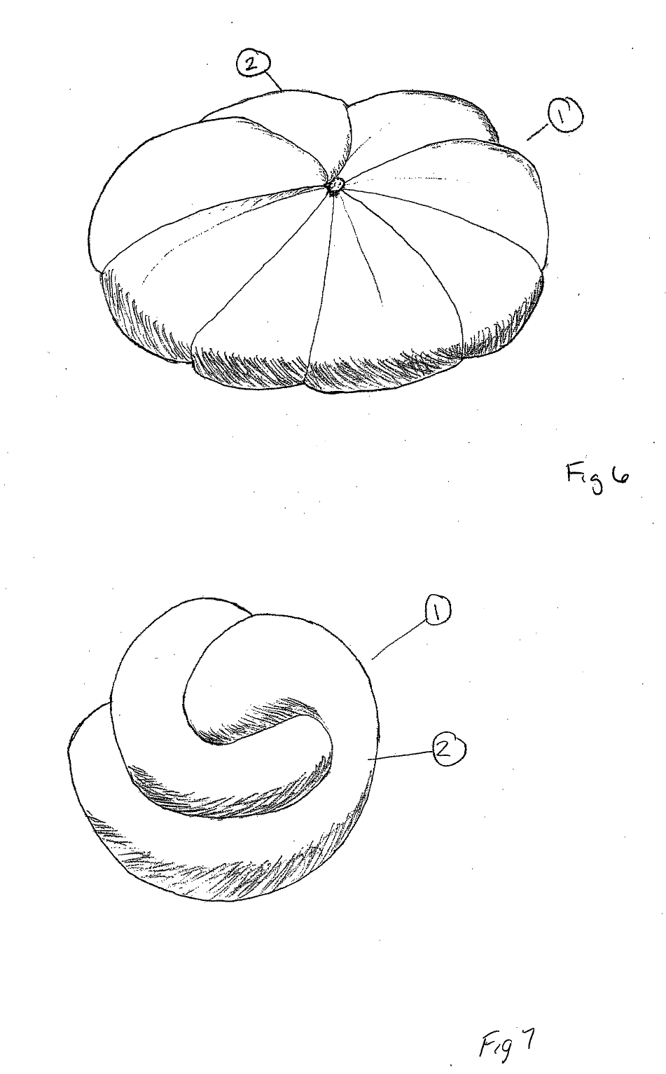

The expandable material (3) is an inert and biocompatible material which will swell and expand the device in the presence of a liquid. Water-soluble polymers such as hydrogels, cellulose, alginates, or hygroscopic materials, are particularly well suited for use as an expandable material in the device, either alone or in combination. Superabsorbent polymers are especially well suited for this application because of their high swelling capacity. Hydrogels suitable for this application may be anionic, cationic, non-ionic, or a combination thereof. Superporous hydrogels may also be suitable for this application based on their rapid swelling and ability to be compressed into a capsule. The intragastric device expands to a size such that it does not enter the intestines through the pylorus during the intended duration of its placement unless it is deactivated. In one embodiment, the device remains in the fundus of the stomach until deactivated. As shown in FIGS. 5A through 7, after swelling, the final shape of the device may adopt various forms including, but not limited, to kidney-shaped, oblong, tomato-shaped (FIG. 6), cylindrical, oval, rectangular, ellipse, triangle, conical, trapezoidal, star-like, pear-like, umbrella-like, butterfly, bow-tie, snare-like, coil, helical, spiral (FIG. 7), doughnut, or spherical form. It is advantageous that the device has a diameter measuring greater than 5 cm in order to prevent the device from migrating out of the stomach.

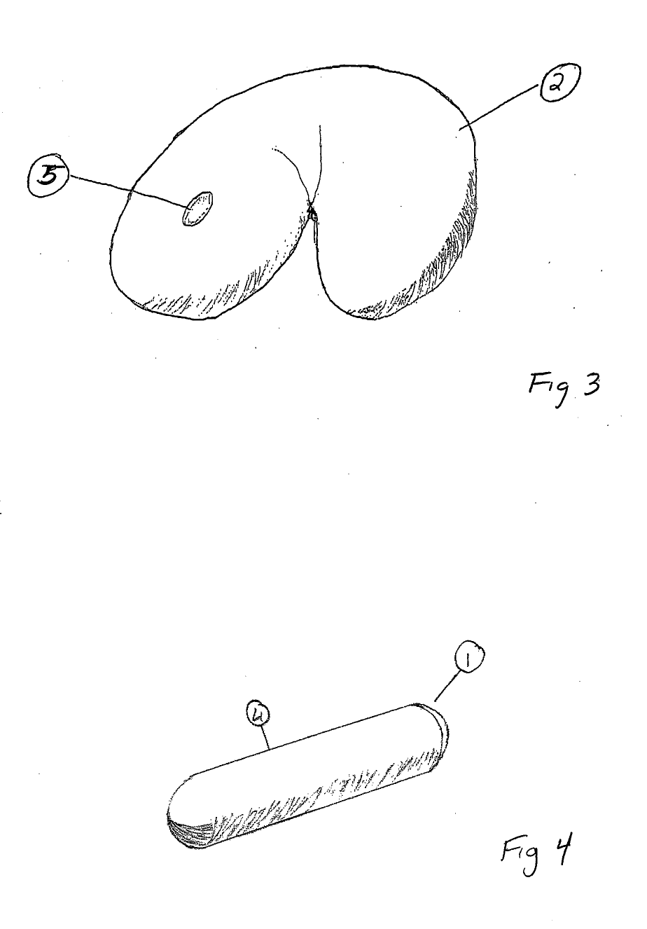

The intragastric device (1) may further comprise at least one controlled deactivation means (5), as illustrated in FIG. 3. Examples of deactivation means include physically rupturable non-degradable membranes (2) and mechanically engineered rupture sites on the non-degradable membranes (2) including, but not limited to, biodegradable rupture portions, ultrasonic rupture sites, magnetic rupture means, electrically-induced mechanisms, chemically-induced mechanisms, mechanically-induced mechanisms, bioabsorbable rupture portions, rupture valves, or other suitable sites as would be known to one of skill in the art. The deactivation means may be of various size and/or shapes and may exist as a single deactivation site or as multiple deactivation sites on the device. It is desired that the device remain in the body of a patient for a determined amount of time. In another embodiment, the intragastric device non-degradable porous membrane is not disruptable under physiological conditions for a period of at least ten days. In a further embodiment, the membrane is not disruptable for a period at least thirty days. By “disruptable” it is meant to break apart or to interrupt the normal course or unity of the membrane. Further, it may be desirable to have one or multiple devices comprising the expandable material delivered to a patient at one time. This may be accomplished via delivery of a single device or multiple devices. In one embodiment, the single device may also comprise multiple individual units enclosed in one membrane. In one embodiment, the intragastric device may comprise at least one non-degradable porous membrane having a swellable material enclosed within the at least one porous membrane to form multiple units, and non-degradable outer shell membrane which contains the multiple units.

In certain embodiments, the non-degradable porous membrane maintains a relative stable pore size during expansion of the material. For instance, the non-degradable membrane may exhibit a pore size in its non-expanded state which is relatively the same or slightly less than the pore size in its expanded state, thereby maintaining the expandable material inside of the device membrane.

The present invention also provides a method of minimally invasive device delivery including swallowable delivery or endoscopic delivery. The in-vivo test device is delivered using an endoscope and then hydrated in situ with hydrating fluid, such as water, after placement in the stomach. The device can then be imaged via x-ray fluoroscopy and/or CT after a suitable period of time to allow for the expandable material to swell or increase the volume of the device. The time required for the device to swell to a functional volume of greater than 5 cm in diameter ranges depending upon the choice of non-degradable membrane and expandable material and packing practice. It is desirable that this period of time be between about 5 minutes (or less) and approximately 2 hours, or more preferably, not longer than 1 hr. The device may then be imaged regularly for visual observations of placement and size.

The following embodiments of the present invention will now be described by way of example only and not intended to limit the scope of the invention.

### EXAMPLE 1

A device was made using an anisotropic ePTFE membrane and approximately 2 grams of hydrogel (BASF, Luquasorb 1010, Florham Park, N.J.). The ePTFE membrane was coated with a glutaraldehyde cross-linked PVA coating to render it hydrophilic. The coated membrane was cut into a 6″×3″ (l×w) rectangle and the hydrogel was placed in the center of the membrane. The membrane was stretched in the transverse direction approximately 50%. The corners of the membrane were brought together and tied into a knot creating a bag. The bag was then placed into a container containing more than 200 ml of tap water and observed. The device swelled to approximately 200 ml within 10 minutes.

### EXAMPLE 2

A device was constructed as in Example 1; however, the coated membrane was not stretched prior to making the bag. The bag was then placed into approximately 200 ml of tap water and observed. The device swelled to approximately 200 ml within 45 minutes.

### EXAMPLE 3

A device was constructed using the method described in Example 1. In addition to the hydrogel, 4 grams of BaSO₄ (Mallinckrodt, MK8821-04, Phillipsburg N.J.) were also placed within the bag for radiopacity. The bag was placed into tap water and observed under fluoroscopy. The device was visible using x-ray fluoroscopy under a 40 mm aluminum plate.

### EXAMPLE 4

A device made per Example 1 was made and placed into a 50:50 solution of tap water and contrast solution (GE Healthcare OMNIPAQUE 300, Princeton, N.J.) and allowed to swell. The device was visible using x-ray fluoroscopy under a 40 mm aluminum plate.

### EXAMPLE 5

A device was made using an anisotropic ePTFE membrane and 5 grams of hydrogel (BASF Luquasorb 1270). The ePTFE membrane was coated with a glutaraldehyde cross-linked PVA coating to render it hydrophilic. The coated membrane was cut into one 10″×7″ (L×W) rectangle and eight 2″×2″ squares. Each smaller square was stretched in the transverse direction approximately 50% and 0.5 grams of the hydrogel was placed in the center of each small membrane. The corners of each small membrane were brought together and tied into a knot creating a small bag. The large square was then stretched in the transverse direction approximately 50% and 1 gram of the hydrogel was placed in the center of the large square membrane. The small bags were then also placed on the center of the large square membrane and the corners of the membrane were brought together and tied into a knot creating a large bag, with smaller bags inside. The device was then placed into 500 ml of tap water and observed.

| Time | Weight |
|---|---|
| (minutes) | (grams) |
| 10 | 52 |
| 15 | 80 |
| 20 | 114 |
| 30 | 209 |
| 45 | 264 |
| 60 | 315 |

### EXAMPLE 6

A device was made using an anisotropic ePTFE membrane, 3 grams of hydrogel (Degussa FP530, Parsippany, N.J.), and 1 gram of bismuth aluminate hydrate (Sigma Aldrich 510289, St. Louis, Mo.). The ePTFE membrane was coated with a glutaraldehyde cross-linked PVA coating to render it hydrophilic. The coated membrane was cut into a 12″×8″ rectangle and stretched to form a 12″×12″ square. The hydrogel and bismuth aluminate hydrate were placed in the center of the membrane. The corners of the membrane were brought together and tied into a knot creating a bag with a 2″ neck (distance from the ball of hydrogel and bismuth powder to the knot). The bag was then placed into 500 ml of tap water and observed.

| Time | Weight | |
|---|---|---|
| (minutes) | (grams) | Shape |
| 5 | 31.2 | |
| 10 | 59.9 | |
| 15 | 157.2 | Firm kidney bean |
| 20 | 228.5 | Ends of kidney bean start |
| | | to overlap |
| 25 | 268.9 | Ends overlapped ~1.5″ |
| 30 | 280.1 | |
| 45 | 310.8 | |
| 60 | 324.2 | |

The device was then imaged with x-ray fluoroscopy under a 40 mm aluminum plate. The device was slightly visible.

### EXAMPLE 7

A device was made using an anisotropic ePTFE membrane, 3 grams of hydrogel (Degussa FP 530), and 0.001″×0.015″ (thickness×width) gold ribbon (California Fine Wire Company, Grover Beach, Calif.). The ePTFE membrane was coated with a gluteraldehyde cross-linked PVA coating to render it hydrophilic. The coated membrane was cut into a 12″×8″ (L×W) rectangle and then stretched to form a 12″×12″ square. The hydrogel was placed in the center of the membrane along with ten (10) 1-2 mm long gold pieces. The corners of the membrane were brought together and tied into a knot creating a bag with a 2.5″ neck. The bag was then placed into 500 ml of tap water and observed.

| Time | Weight | |
|---|---|---|
| (minutes) | (grams) | Shape |
| 5 | 15.7 | |
| 10 | 26.6 | |
| 15 | 47.5 | |
| 20 | 74.4 | Loose, conformable |
| 30 | 152.4 | Firm, conformable |
| 45 | 262.1 | Firm, conformable |

The device was then imaged with x-ray fluoroscopy under a 40 mm aluminum plate. The gold particles could be seen.

### EXAMPLE 8

A device was made per Example 7. The device was then placed in a solution of 500 grams of warm tap water and 50 grams of table sugar and observed.

| Time | Weight |
|---|---|
| (minutes) | (grams) |
| 5 | 17.6 |
| 10 | 22.5 |
| 15 | 33.3 |
| 20 | 39.5 |
| 25 | 50.1 |
| 30 | 87.4 |
| 35 | 136.8 |
| 40 | 182.4 |
| 45 | 218.9 |
| 50 | 245.1 |
| 60 | 277.1 |
| 70 | 284.4 |

### EXAMPLE 9

A device was made using an isotropic ePTFE membrane and 4 grams of hydrogel (Degussa FP 530). The ePTFE membrane was coated with a glutaraldehyde cross-linked PVA coating to render it hydrophilic. The coated membrane was cut to a 12″×12″ square and the hydrogel was placed in the middle. The corners were then gathered together and a knot was tied as high up as possible. The device was then placed into warm tap water and observed.

| Time | Weight | |
|---|---|---|
| (minutes) | (grams) | Shape |
| 5 | 23.5 | Amorphous bag, loose |
| 10 | 53.5 | Loose amorphous bag |
| 15 | 111.9 | Loose disc shaped bag |
| 20 | 167 | Loose bag |
| 25 | 242.1 | Loose bag |
| 30 | 301 | Loose bag |
| 35 | 387 | Loose bag |
| 40 | 477.8 | Loose bag |
| 45 | 571 | Loose bag |

### EXAMPLE 10

A device was made using an isotropic ePTFE membrane in accordance with U.S. Pat. No. 7,306,729, 4 grams of hydrogel (Degussa FP 530), and 1 gram of bismuth aluminate hydrate (Sigma Aldrich). The ePTFE membrane was coated with a glutaraldehyde cross-linked PVA coating to render it hydrophilic. Four pieces of coated membrane were each cut to a 12″×12″ square. One (1) gram of the hydrogel was placed in the middle of the first piece of membrane, and then the next piece of membrane was placed on top of the hydrogel. This was repeated until all four pieces were on top of each other, each with 1 gram of hydrogel. The corners were all then gathered and tied into a knot. The distance from the ball to the knot was approximately 1.75″. The device was then placed into warm tap water and observed.

| Time | Weight | |
|---|---|---|
| (minutes) | (grams) | Shape |
| 5 | 27.7 | Loose egg shape |
| 9 | 60.2 | Firm tomato shape. |
| | | Distinct hydrated/non- |
| | | hydrated layers |
| 12 | 81.5 | Intentionally burst outer |
| | | membrane |
| 24 | 67.1 | Firm tomato shape. |
| | | Distinct hydrated/non- |
| | | hydrated layers |
| 30 | 83 | Firm egg shape |
| 35 | 86.4 | Firm egg shape |
| 49 | 92 | |

### EXAMPLE 11

A device was made per Example 17, except that instead of just a knot to form the bag, the bag was sealed using an ePTFE fiber tied around the top. The excess material above the fiber was then tied into a knot and some Loctite 4013 (Henkel North America, Rocky Hill, Conn.) was added to the knot. The device was then placed in warm water and observed. Similar results were obtained as above. Final size was 404 grams and the device was tomato shaped. The device was then placed into an environmental chamber @ 37° C. & 75% RH. An approximately 3 lb weight was placed on top of the device. After 96 hours, the device was removed from the chamber. The weight of the device upon removal was 264.4 grams. The device was then placed in warm tap water. After 7 minutes, the weight of the device was 383.7 grams.

### EXAMPLE 12

A device was made using an ePTFE tube (1″ diameter×0.003″ wall thickness) and 3.5 grams of hydrogel (BASF Luquasorb 1270). The ePTFE tube was coated with a glutaraldehyde cross-linked PVA coating to render it hydrophilic. The tube was cut to approximately 11″ and the hydrogel was placed into it. The ends were sealed with wire terminal ends crimped on them. The device was placed in warm water and observed.

| Time | Weight |
|---|---|
| (minutes) | (grams) |
| 5 | 97.5 |
| 10 | 111 |
| 15 | 125.7 |
| 20 | 147 |
| 25 | 161 |
| 30 | 185 |
| 35 | 193 |
| 40 | 206 |
| 45 | 211 |
| 50 | 235 |
| 55 | 282 |
| 60 | 294 |

### EXAMPLE 13

A device was made using an anisotropic ePTFE membrane and approximately 2 grams of hydrogel (BASF Luquasorb 1270). The ePTFE membrane was coated with a glutaraldehyde cross-linked PVA coating to render it hydrophilic. The coated membrane was cut into a 12″×8″ (L×W) rectangle and the hydrogel was placed in the center of the membrane. The membrane was stretched in the transverse direction approximately 50%. The corners of the membrane were brought together and tied into a knot creating a bag. The device was placed in warm tap water and allowed to reach full size. A piece of 1.5″ long×0.004″ diameter wire (Fort Wayne Metals, MP-DFT-25% Ag, Fort Wayne, Ind.) was placed onto the device. The wire was then connected to a 9V battery. The wire immediately (<1 second) glowed orange. The battery was then disconnected and the device was observed. There was approximately ¾″ slit in the device under the wire that allowed the hydrated hydrogel to leak out of the device.

### EXAMPLE 14

A device was made per Example 13. The device was then placed into an aluminum mold and then pressed using a dowel and Arbor press to form a pill-shaped form.

### EXAMPLE 15

A device was made per Example 13. The device was then rolled into a tight cylinder and placed into a gelatin capsule (Spectrum Pharmacy Products C1716, Tucson, Ariz.).

### EXAMPLE 16

A device was made per Example 13. The device was then placed into an aluminum mold and pressed using a dowel with a convex end into a pill shape and then sprayed or dip coated with a 3% solution of poly(acrylic acid) (Sigma Aldrich, 323667, St. Louis, Mo.), then dried in an oven at 100° C. to form a coated hydrogel capsule.

### EXAMPLE 17

A device was made using an isotropic ePTFE membrane and 8 grams of hydrogel (Degussa FP 530). The ePTFE membrane was coated with a glutaraldehyde cross-linked PVA coating to render it hydrophilic. The coated membrane was cut to an 11″×11″ square and the hydrogel was placed in the middle. The device was transferred into a metal cylindrical tube having dimensions: H=4″ (10.16 cm), and D=0.85″ (21 mm). The device was allowed to drop to the bottom of the cylinder. The corners were then gathered together and a knot was tied at the top of the tube, and sealed with a PTFE fiber knot and Loctite 4013 adhesive. The device was then placed into warm tap water and observed.

| Time | Weight |
|---|---|
| (minutes) | (grams) |
| 5 | 270 |
| 12 | 336 |
| 21 | 361 |
| 36 | 374 |
| 46 | 385.9 |
| 67 | 397.1 |
| 77 | 402.9 |
| 107 | 410.3 |
| 19 Hrs 32 min | 421.4 |
| 91 Hrs | 462.7 |

### EXAMPLE 18

Several devices were made per Example 1, using an anisotropic ePTFE membrane and approximately 2 grams of hydrogel (BASF, Luquasorb 1010, Florham Park, N.J.). The ePTFE membrane was coated with a glutaraldehyde cross-linked PVA coating to render it hydrophilic. The coated membrane was cut into a rectangle measuring 7″ W×10″ L and then transversally expanded to 10″ W×10″ L. Hydrogel powder was placed in the center of expanded membrane. The corners of the membrane were brought together and tied into a knot 2″-2.5″ above the hydrogel, creating a bag.

Additionally, a concentrated solution of Simulated Gastric Fluid (SGF) was prepared as outlined below:

### Preparation of Simulated Gastric Fluid (SGF):

The prepared solution had a pH=1.37.

Simulated Gastric Fluid (SGF), for simulating gastric environment, was made according to the USP method by mixing the following ingredients:

| | | |
|---|---|---|
| 4 | g | NaCl (Fluka) |
| 6.4 | g | pepsin (Mallinckrodt Chemicals) |
| 14 | ml | 12.1M HCl |
| 2 | liters | distilled water |

These devices were placed in the following media and observed:

Warm tap water [40° C.]
4.75% SGF solution
25% SGF solution
100% SGF solution

| | Time | Weight |
|---|---|---|
| Media | (minutes) | (grams) |
| Warm tap water | 5 | 19 |
| | 10 | 57 |
| | 15 | 140 |
| | 30 | 189 |
| | 60 | 203 |
| 4.75% SGF | 5 | 27 |
| solution | 30 | 171 |
| | 90 | 177 |
| 25% SGF solution | 5 | 19 |
| | 10 | 92 |
| | 15 | 108 |
| | 30 | 112 |
| | 60 | 113 |
| 100% SGF | 5 | 36 |
| solution | 13 | 57 |
| | 20 | 54 |
| | 25 | 58 |
| | 30 | 56 |

### EXAMPLE 19

Two devices were made per Example 17, with the addition of three 12″ gold ribbons (0.001″×0.015″ thickness×width) glued to the film using Loctite 4013. The gold ribbon was placed on the film creating 3 intersecting rings for the ability to image via x-ray fluoroscopy. One device was placed in warm tap water (data below) and the other was tested in a canine model. The in-vivo test device was delivered using an endoscope and hydrated with approximately 1 liter of warm bottled water for about 45 minutes. The size of the swollen device was then endoscopically examined. Hydration of the device was continued for another 45 minutes with 1 liter of warm (T˜37° C.) bottled water. The device was then imaged via x-ray fluoroscopy and CT after approximately 1 hour. The device was then imaged every other day for 7 days. After 7 days, the device was visually observed using an endoscope and retrieved. The device remained in the stomach for the duration of the study. Upon removal, the device was approximately 100 ml in size and was observed to contain several “air” bubbles within the membrane. The hydrogel was then cultured for gas forming bacteria; cultures were negative.

| Time | Weight | |
|---|---|---|
| (minutes) | (grams) | Shape |
| 5 | 12.3 | |
| 10 | 22.5 | |
| 15 | 50.2 | |
| 20 | 143.2 | |
| 25 | 234.6 | |
| 30 | 326.7 | Tomato |
| | | shaped |
| 35 | 369.9 | Getting firm |
| 40 | 407.4 | Getting firm |
| 45 | 415.9 | Firm |
| 50 | 424.6 | Firm |
| 55 | 429.7 | Firm |

### EXAMPLE 20

A device was made per Example 17, with the addition of three 12″ gold ribbons (0.001″×0.015″ thickness×width). The gold ribbon was placed on the outside of the device by encapsulating it with a 0.090″ wide film of EFEP. The captured gold ribbon created 3 intersecting rings for the ability to image via x-ray fluoroscopy. The device was tested in a canine mode per Example 19. The device remained in vivo for 32 days, after which it was safely excreted. Upon examination of the excreted device, it was discovered that the membrane had developed a small tear near the knot, which allowed the contents of the device to leak out thereby reducing the volume and size of the device.

### EXAMPLE 21

Two pieces of isotropic ePTFE membrane were cut into 6″ squares and a 5.25″×5.75″ (ID×OD) ring of 0.001″ thick EFEP was cut. The EFEP ring was centered onto the first piece of membrane. Six (6) grams of hydrogel was then placed onto the center of the first piece of membrane, and then the second piece of membrane was placed over the EFEP ring, first piece of membrane, and hydrogel. A soldering iron, heated to 830° F., with a blunt tip was used to melt the EFEP into the two pieces of membrane, creating a ⅛″-¼″ ring defining the perimeter of the device.

### EXAMPLE 22

A device was constructed per example 21, with an additional 0.001″ thick EFEP 1.25″×1.75″ (ID×OD) ring. The smaller EFEP ring was placed concentric to the first EFEP ring onto the first membrane and 4 grams of hydrogel were placed between the two rings of EFEP. A modified soldering iron at 600 F was used to melt the EFEP rings and bond the layers. The excess membrane from the center of the device was removed to create a donut.

1. An intragastric device comprising a non-degradable porous membrane enclosing a material which expands upon contact with fluid, and causes the volume of the device to increase.

2. The intragastric device of claim 1 wherein the non-degradable porous membrane is water permeable.

3. An intragastric device of claim 1 wherein said device is sized to be delivered orally.

4. An intragastric device of claim 2 wherein said device is sized to be delivered orally via a swallowable capsule.

5. The intragastric device of claim 1 further comprising at least one controlled deactivation means.

6. An intragastric device of claim 1 wherein the non-degradable porous membrane maintains a constant relative pore size during expansion of the material.

7. An inflatable gastric device comprising a non-degradable fibrillated membrane enclosing an expandable material wherein the expandable material causes the device to expand upon contact with gastric liquids.

8. An inflatable gastric device of claim 7 wherein said inflatable gastric device is sized to be delivered orally.

9. An inflatable gastric device of claim 6 wherein said fibrillated membrane is not disruptable under physiological conditions for a period of at least ten days.

10. The inflatable gastric device of claim 6 further comprising a rupture valve.

11. An intragastric device comprising:
a. a non-degradable porous membrane, and
b. a swellable material enclosed within the porous membrane to form an initial shape wherein said initial shape expands to an expanded volume upon contact with liquids to form a final shape.

12. The intragastric device of claim 11 wherein the final shape has at least 5 times volume of initial shape.

13. The intragastric device of claim 11 wherein the final shape has about 2-3000 times volume of initial shape.

14. The intragastric device of claim 11 wherein the initial shape has volume between 0.1 ml to 28 ml.

15. The intragastric device of claim 11 wherein the initial shape has volume between 0.5 ml to 10 ml.

16. The intragastric device of claim 11 wherein the final shape has volume between 20 ml to 1500 ml.

17. The intragastric device of claim 11 wherein the final shape has volume between 100 ml to 500 ml.

18. The intragastric device of claim 11 wherein said initial shape is selected from kidney, oblong, cylindrical, oval, rectangular, ellipse, triangle, conical, trapezoidal, star-like, pear-like, umbrella-like, butterfly, bow-tie, snare-like, coil, helical, spiral, doughnut, or spherical shape configuration.

19. The intragastric device of claim 11 wherein the final shape is selected from kidney, oblong, cylindrical, oval, rectangular, ellipse, triangle, conical, trapezoidal, star-like, pear-like, umbrella-like, butterfly, bow-tie, snare-like, coil, helical, spiral, doughnut, or spherical shape configuration.

20. The intragastric device of claim 11 wherein said final shape has a different configuration than said initial shape.

21. The intragastric device of claim 11 further comprising a contrast agent.

22. The intragastric device of claim 11 wherein a portion of the device is radiopaque.

23. An intragastric device comprising:
a. at least one non-degradable porous membrane,
b. a swellable material enclosed within the at least one porous membrane to form multiple units, and
c. non-degradable outer shell membrane which contains the multiple units.
